(19) United States
(12) Patent Application Publication
(10) Pub. No.: US 2015/0073332 A1
(43) Pub. Date: Mar. 12, 2015

(54) FLEXIBLE STRETCHABLE TROCAR TO FACILITATE SINGLE INCISION LAPAROSCOPIC SURGERY

(71) Applicant: Robert C. Knowles, Peterborough, NH (US)

(72) Inventor: Robert C. Knowles, Peterborough, NH (US)

(21) Appl. No.: 14/019,686

(22) Filed: Sep. 6, 2013

(51) Int. Cl. A61B 17/34

(52) U.S. Cl. CPC A61B 17/3439
USPC 604/26

(57) ABSTRACT

A headless, flexible, two-part, semi-flexible trocar is formed from two semi-circular sections to form a hollow tubular body having a flared proximal end and a tapered distal end. The sections may temporarily move with respect to one another to increase the diameter of the tubular structure. A top portion protruding beyond the body may readily be trimmed as desired to allow a greater range of motion for an optical or surgical instrument within the body cavity. A valve/fulcrum within the tubular structure helps control the optical or surgical instruments. When tissue needs to be removed from the body cavity, the stem of the trocar may be expanded to allow passage of the tissue being removed, which stretches the incision. The insufflation gas is injected into the body cavity at a point other than through the trocar, which further optimizes intraoperative optics while minimizing tissue trauma.

### CROSS-REFERENCES TO RELATED APPLICATIONS

(NOT APPLICABLE)

### STATEMENT REGARDING FEDERALLY SPONSORED RESEARCH OR DEVELOPMENT

(NOT APPLICABLE)

### BACKGROUND OF THE INVENTION

The invention pertains to trocars and, more particularly, to flexible, low to no profile, two-piece, expandable trocars facilitating single incision laparoscopic surgery (SILS) allowing both instrument insertion/egress and having access for achieving pneumoperitoneum.

Laparoscopic surgical techniques are both well known and widely practiced for performing a wide variety of surgical procedures. The major advantage of laparoscopic procedures is that no large incision needs to be made into a patient, thereby greatly reducing patient recovery time and typically, post-operative pain. In some cases, simple procedures performed laparoscopically may be done either on an outpatient basis, or with a limited hospitalization. SILS limits these smaller incisions to a single incision at the umbilicus. SILS is further pushing what typically has been an inpatient procedure to be completed as an outpatient basis. Such procedures previously typically required a multi-day hospitalization when conventional surgical techniques were used.

Laparoscopic surgery typically utilizes multiple trocars through multiple small incisions for the insertion of a camera and surgical instruments, as well as introduction of materials such as sutures, repair meshes, and the like required for the specific surgical procedures. One or more additional trocars may be used to inflate the abdomen or other body cavity to facilitate the surgery being performed. The camera provides an image on a monitor which is used by the surgeon to guide his or her manipulation of the instruments.

It has been observed that patient discomfort is proportional to the diameter of the trocars utilized for the surgery, large diameter trocars resulting in more discomfort, and small diameter trocars resulting in less discomfort. It has also been recognized that a puncture or incision made by a small diameter, for example, a 5 mm trocar may be virtually self healing, requiring no suture to close the puncture or incision (i.e., fascial defect) upon withdrawal of the trocar. This provides additional incentive to utilize small diameter trocars whenever possible.

Conventional trocars utilized for laparoscopic procedures are typically substantially rigid and include a head disposed at the proximal end of a stem or shaft. A port and sometimes a valve are included to allow insufflation of the abdomen (i.e., the inflation with carbon dioxide or a similar gas). Insufflation of the abdomen during laparoscopic surgery creates a working space for visualization and performing surgery. While the abdominal cavity has been chosen to illustrate the use of the novel trocar of the invention, it will be noted that the novel trocar may be used in other body cavities as well.

An opening in the trocar head allows the insertion of an optical device (e.g., a camera), surgical tools, or materials. However, the rigid head and the fixed diameter of conventional trocars present several problems. In particular, in SILS, the necessity of placing two 5 mm large-headed trocars in close proximity inserted through a single incision severely restricts movements of instruments inserted therethrough and interferes with successful completion of surgical procedures. A single millimeter difference in range of motion at the umbilicus translates to centimeters in range of motion at the operative site based on fulcrum mechanics. One problem is that rigid adjacent large-headed trocars contact one another, thereby limiting range of motion and severely restricting surgical instrument movement.

In addition, the introduction of gas at the primary port (i.e., one of the adjacent trocars of the prior art) may interfere with clear laparoscopic imaging necessary for safety.

It would, therefore, be desirable to provide a trocar that would provide improved access to a body cavity for performance of laparoscopic surgery and would move the gas insertion point away from the incision and working area.

### BRIEF SUMMARY OF THE INVENTION

In an exemplary embodiment, a flexible expandable trocar includes a pair of semicircular pieces fitted one into another to obtain a required range of diameters. No head is provided. Rather, a flared end facilitates insertion of surgical instruments and/or optical elements into the body cavity. A top portion protruding from the body may readily be trimmed substantially flush with the patient's skin to allow the surgeon even greater range of motion for the surgical instruments. When tissue needs to be removed from the body cavity, the stem of the trocar may be temporarily expanded to allow passage of the tissue being removed. The expandability of the novel trocar design also allows the insertion of surgical instruments larger than the 5 mm instruments typically used. This provides the surgeon access to all available laparoscopic instrumentation to safely and efficiently complete the intended laparoscopic intervention (i.e., 10 mm graspers, laparoscopic staplers, specimen retrieval pouches). This temporary expansion may stretch the incision minimizing the requisite larger incision of the larger diameter trocar of the prior art. The insufflation gas may be injected into the body cavity at a different point than through the trocar. This is done using a device similar in construction to an “angiocath.” The size of the opening in the body wall left by this device is so small that no stitches are required at the completion of the surgery. No suturing translates to less wound complications, less cost, and greater intraoperative efficiency. At these puncture sites, patients rarely even realize that an additional body intrusion has taken place, and no post operative pain has been reported. Further, the performance of optical instruments benefits from moving the insufflation gas port away from the trocar as fogging and other effects caused by inserting insufflation gas at the trocar are eliminated.

It is therefore an object of the invention to provide a trocar that allows a greater range of movement for surgical instruments and/or optical elements.

It is another object of the invention to provide a headless trocar that allows insufflation gas to be inserted away from the trocar.

It is still another object of the invention to provide a trocar formed from two semicircular pieces that fit one within the other.

It is an additional object of the invention to provide a trocar whose stem may temporarily be diametrically expanded to facilitate removal of tissue from a surgical site and insertion and removal of large diameter instruments that may not require an enlargement of the incision.

It is a further object of the invention to provide an improved trocar having a fulcrum within its stem and away from the head to improve manipulability of surgical instruments, optimizing the fulcrum advantage of single incision surgery.

It is a still further object of the invention to provide an improved trocar using a tiny insufflation port inserted into a patient away from the trocar.

It is still further object of invention that a slotted temporary cannula conducts the insufflation catheter through the abdominal wall allowing its removal while leaving the insufflation catheter in place.

It is an object of invention that the bivalve or quad valve mechanism of the trocar is of a sponge nature to cleanse the optical lens and to apply an anti-fog liquid to the lens.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a side elevational, schematic view of a trocar and obturator of the prior art;

FIG. 2a is a perspective view showing two semicircular pieces poised one above the other in a position to be formed into a trocar stem in accordance with the invention;

FIG. 2b is an end elevational, schematic view of the semicircular pieces of FIG. 2a;

FIG. 2c is an end elevational, schematic view of the two semicircular pieces of FIG. 2a formed into an expandable circular structure;

FIG. 3a is a side elevational view of a trocar in accordance with the invention formed from two semicircular pieces;

FIG. 3b is an end elevational view of the trocar of FIG. 3a;

FIG. 3c is a perspective schematic view of the two halves of the trocar of FIG. 3a showing a flared end thereof and prior to assembly;

FIG. 3c′ is a perspective schematic view of the two halves of the trocar of FIG. 3c assembled into a complete trocar;

FIG. 3d is a side elevational, schematic view of ribs of the trocar of FIG. 3a disposed in a spiral configuration;

FIGS. 3e and 3f are top plan, schematic views of two embodiments of a valve/fulcrum of the trocar of FIG. 3a;

FIGS. 3g and 3h are side elevational, schematic views of two additional alternate embodiments of a valve/fulcrum of the trocar of FIG. 3a;

FIG. 4 is a side elevational view of the trocar of FIG. 3a with an obturator inserted therein;

FIG. 5a is a simplified schematic system diagram of an arrangement suitable for injecting insufflation gas into a body cavity away from the trocar of FIG. 3a;

FIGS. 5b and 5c show alternate embodiments of an inserter that may be split along its major axis for removal from a catheter; and

FIG. 6 is a schematic plan view of an abdominal region of a patient with a pair of trocars inserted through the umbilicus and with a remotely located gas insertion port.

### DETAILED DESCRIPTION OF THE INVENTION

The disadvantages of one-piece, rigid, headed trocars of the prior art have been discussed hereinabove. The trocar of the invention overcomes all of the disadvantages presented by such prior art trocars. The novel trocar is formed from two semicircular sections that mate to form a trocar whose diameter is temporarily expandable as the two semicircular sections move with respect to one another.

Further, the elimination of the trocar head allows a greater range of movement for surgical instruments and/or optical elements, thereby allowing access to a larger surgical field. An excess length of the trocar extending outside a patient's body may, when desired, readily be trimmed to allow even more range of motion for surgical instruments.

The elimination of the trocar head with its gas port yields the advantage that the insufflation gas may be injected into the body cavity of interest remotely from the site of the trocar. This eliminates the problem of fogging of optical elements caused when insufflation gas is injected through the stern of the trocar as is done in trocars of the prior art.

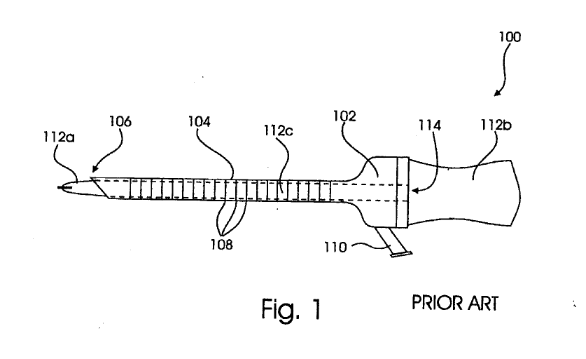

Referring first to FIG. 1, there is shown a side elevational view of a trocar of the prior art, generally at reference number 100. Trocar 100 has a head 102 coaxially attached to a hollow stem 104 having a distal tip 106. A series of ridges 108 are disposed on an outer circumference of hollow stem 104. A gas injection port 110 forms a part of the head 102. An obturator having a tip 112a, a handle 112b, and a shaft 112c is shown inserted in trocar 100. A central opening 114 in head 102 allows access to the hollow interior, not specifically identified, in stem 104.

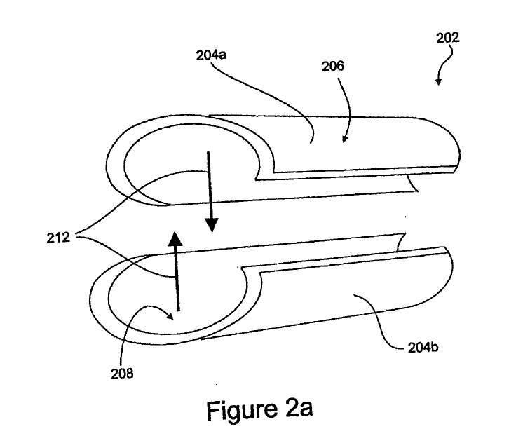

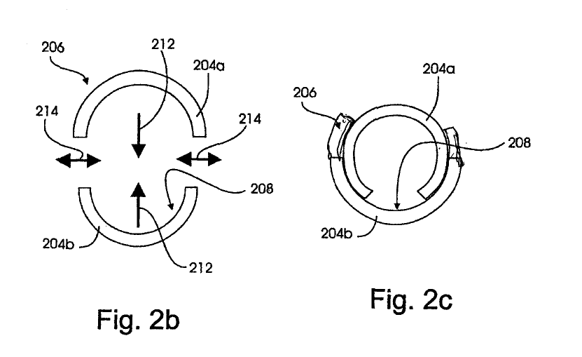

FIG. 2a shows a perspective view with two semicircular sections 204a, 204b poised one above the other in a position to be formed into a trocar stem in accordance with the invention, generally at reference number 202. An upper semicircular section 204a is poised above a lower semicircular section 204b, semicircular section 204b being inverted with respect to semicircular section 204a. Arrows 212 indicate the direction of movement of semicircular sections 204a, 204b toward each other. When engaged, major outside surface 206 of semicircular section 204a contacts major interior surface 208 of semicircular section 204b. The sections 204a, 204b are generally formed of a thin polymeric material, although other materials may be suitable, and the invention is not meant to be limited to a particular material, which is flexible and resilient to provide for slight expansion while enabling retraction to their original shapes.

FIGS. 2b and 2c show end elevational, schematic views of the semicircular pieces 204a, 204b of FIG. 2a poised one above the other, and engaged with one another, respectively. In addition to arrows 212 indicating the direction of vertical motion, arrows 214 indicate the direction of horizontal motion (i.e., the squeezing compression of one of semicircular sections 204a, 204b).

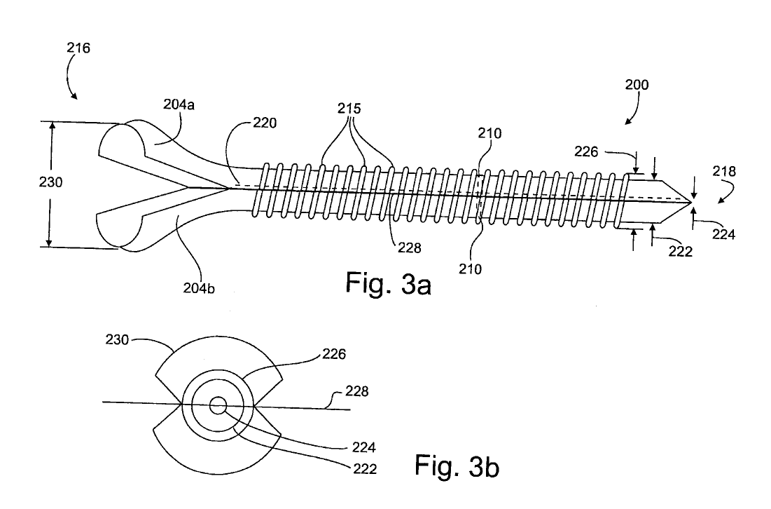

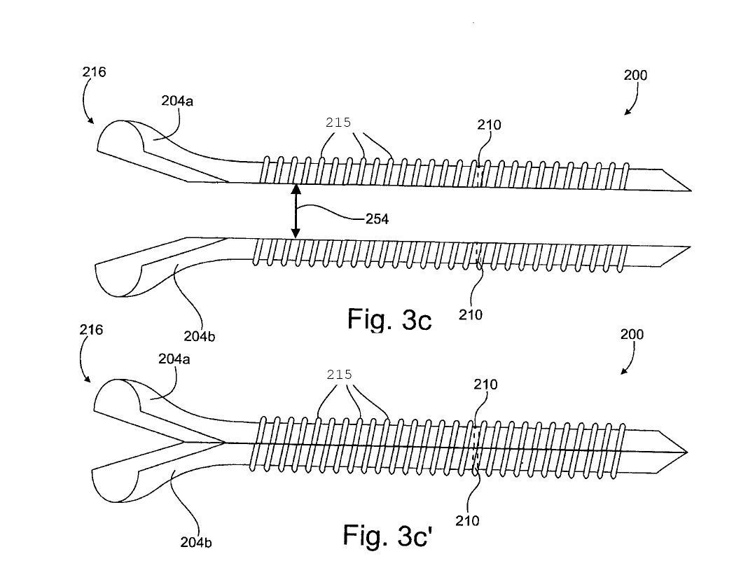

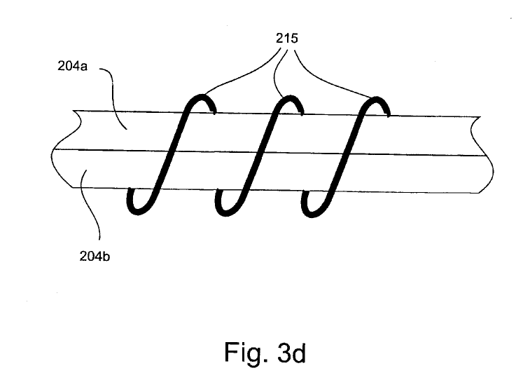

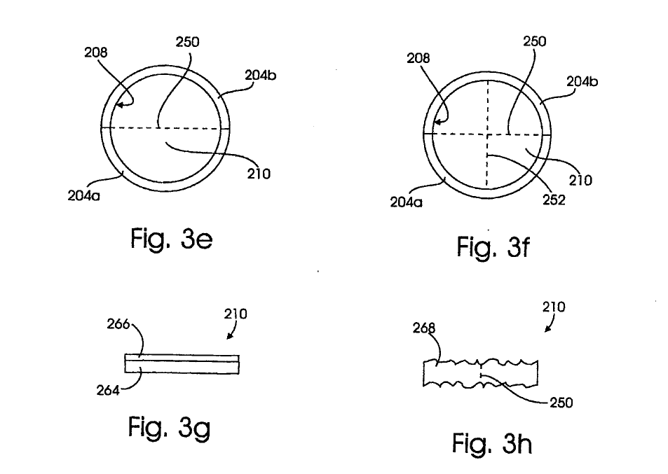

As seen in FIG. 2c, semicircular section 204a has been compressed and inserted into semicircular section 204b such that major outer surface 206 engages major inner surface 208. As semicircular pieces 204a, 204b are made from a resilient material and both have relatively thin cross sections, material memory of the compressed semicircular section 204a creates an outward pressure against major inside surface 208. The surfaces 206, 208 may circumferentially slide against each other when pressure is exerted on major inside surface 208 and an inside surface of semicircular section 204. Such pressure may be exerted from inside the stem of a trocar 200 (FIG. 3a) when tissue must be withdrawn from within a body cavity or when a larger tool such as a stapler or the like is inserted through the trocar 200. That is, when assembled, the trocar stem may be sized to accommodate a 5 mm tool or camera or the like. There are times during a surgical procedure when a larger tool must be inserted through the trocar or when tissue or the like must be extracted, and rather than inserting a larger diameter trocar, the expandable trocar of the invention is capable of accommodating the larger object, for example a stapler having a diameter of 10 mm. As the object is inserted into the trocar, the semicircular pieces 204a, 204b separated slightly to expand the inside diameter of the trocar, while maintaining the insufflated gas defining the surgical working space. When the object is removed, the resilient nature of the semicircular pieces 204a, 204b causes the pieces to contract on themselves and return to the original diameter. In an abdominal procedure especially, the abdominal wall is resilient and capable of slight expansion so that a wider incision will not be necessary. In the event that the semicircular pieces 204a, 204b are inadvertently separated, the abdominal wall or the like will contain the pieces so that the procedure can be finished and the trocar(s) can be safely removed. The larger diameter extractions and insertion of larger diameter tools such as a 10 mm staple typically occur at the very end of surgery, and separation of the semicircular pieces 204a, 204b would be of no consequence.

FIG. 3a shows a side elevational, schematic view of the trocar including a flared proximal end 216 and a narrowed tip 218. FIGS. 3c and 3c′ show a perspective schematic view of two halves 204a, 204b of trocar 200 of FIG. 3a prior to assembly. The two trocar halves 204a, 204b are joined by moving the two halves 204a, 204b together as shown by arrow 254. FIG. 3c′ shows trocar 200 after halves 204a, 204b are assembled. FIGS. 3c and 3c′ show more detailed views of flared proximal end 216 of trocar 200. The flared design is important to assist the surgeon with “threading” a long 5 mm instrument to and through a small orifice.

A series of ribs 215 are disposed circumferentially around the outside surface of the stem formed from semicircular sections 204a, 204b. Ribs 215 may be disposed either parallel to one another or at an acute angle compared to an axis perpendicular axis to the major axis of trocar 200 to one another. In other embodiments, ribs 215 may be continuous spirals. FIG. 3d is a side elevational, schematic view of ribs 215 disposed in a spiral pattern along a portion of trocar 200. The ribs 215 generally serve to secure the trocar in place during the procedure by engaging the abdominal wall or the like. With the spiral pattern, the trocar may be rotated to adjust its position in the patient.

A demarcation line 228 shows the break between semicircular sections 204a, 204b. A line 220 shows one possible location of an edge of semicircular section 204b inserted into semicircular section 204a.

A valve/fulcrum 210 is disposed within a hollow region, not specifically identified, of the stem of trocar 200 perpendicular to the major axis thereof. Valve/fulcrum 210 is typically formed from a thin, resilient, impermeable material and is typically disposed approximately between flared proximal end 216 and tip 218 of the trocar 200. It will be recognized that valve/fulcrum 210 may be placed elsewhere along the major axis of trocar 200 to meet a particular operating requirement.

Valve/fulcrum 210 serves two major purposes. First, valve/fulcrum 210 serves as at least a partial seal to minimize outflow of the insufflation gas from the body cavity into which trocar 200 is inserted. That is, the valve extends across the interior channel in the trocar to prevent gas outflow. Its second function is to provide a fulcrum that assists a surgeon in controlling surgical instruments inserted through trocar 200 into the body cavity.

Valve/fulcrum 210 may be implemented in several manners. In a first embodiment (see FIG. 3e), a thin flap is formed in two sections, a first of which is attached to an inside surface 208 of semicircular piece 204b, a second of which is attached to an analogous inside surface (not specifically identified) of semicircular piece 204a. Line 250 shows the split between two sections of valve/fulcrum 210.

One important design consideration for valve/fulcrum 210 is that it not “slime” the tip of an optical element inserted into the body cavity. Such “sliming” regularly occurs by current trocar designs when residue builds up on valve/fulcrum 210 from surgical instruments being withdrawn from the body cavity therethrough stalling and interrupting surgical progress. Safe laparoscopic surgery is predicated on the quality of visualization, the same as driving a car. One solution (see FIG. 3g) to the “sliming” problem may include using a two-layer structure for valve/fulcrum 210 wherein a fabric layer 266 is added to the thin, resilient, impermeable layer 264 whose sole function is to wipe the end of an optical instrument as it passes inwardly (i.e., toward the body cavity). Yet another novel solution to the “sliming” problem is to form valve/fulcrum 210 from a sponge or sponge-like material 268 (see FIG. 3h). The sponge material 268 may be treated either at the time of manufacture or at the time of use with an anti-fog or other chemical treatment to help improve the functioning of any optical element inserted into a body cavity through trocar 200. In an alternate embodiment (see FIG. 3f), an additional split 252 is added to the first split 250.

The diameters of various sections of trocar 200 may be seen in FIG. 3a. The diameter of tip 218 is shown at reference number 224, the diameter of the sleeve at reference number 222, the outside diameter of ribs 215 at reference number 226, and the diameter of flared proximal end 216 at reference number 230. The relationship of these diameters may readily be seen in FIG. 3b. It will also be noted in FIG. 3b that ribs 215 may not extend over the entire surface of semicircular section 204a, 204b. Rather, edge portions of semicircular sections 204a, 204b may be devoid of ribs 215 to facilitate the mating of the two semicircular sections 204a and 204b. Generally, the ribs 215 assist in securing the trocar within the body wall.

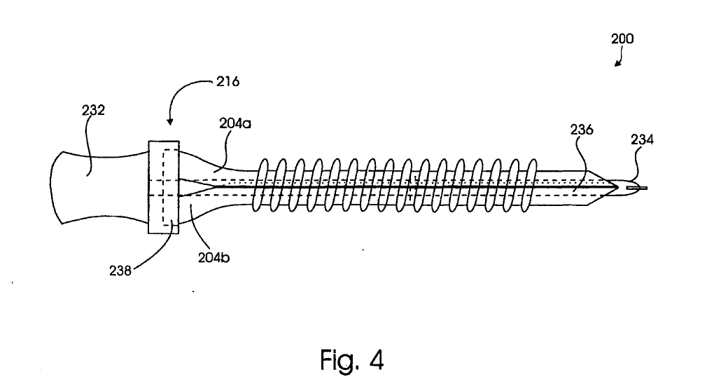

FIG. 4 shows a side elevational, schematic view of the trocar 200 of FIG. 3a with an obturator inserted therein. The obturator has a handle 232, a tip 234, and a shaft 236. Handle 232 has a recess 238 formed therein, sized and configured to accept an outer edge of flared proximal end 216 of trocar 200 therewithin. By capturing the outer edges of flared proximal end 216, more stability is provided to trocar 200, especially as it is inserted into a body cavity. Conventional practice is to insert trocars into body cavities with obturators in place as shown in FIG. 4.

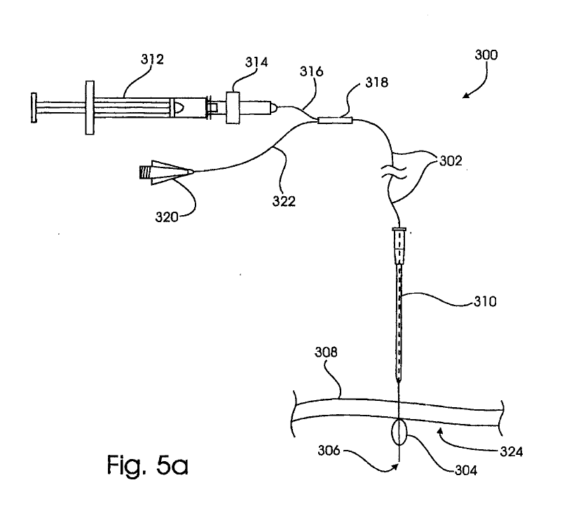

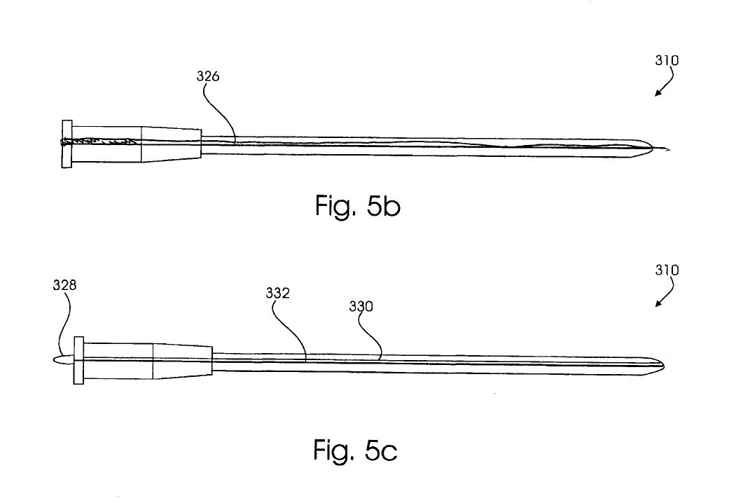

FIG. 5a shows a simplified schematic system diagram of an arrangement suitable for injecting insufflation gas into a body cavity remotely from trocar 200, generally at reference number 300. Apparatus 300 is similar to an angiocath believed to be well known to persons of skill in the medical arts. A thin, biluminal catheter 302 having a balloon 304 proximate its distal end 306 is adapted for insertion through the wall of a body cavity 308, for example, the abdominal cavity of a patient, typically using an inserter 310. Balloon 304 is selectively inflated and deflated by an inflation syringe 312 in combination with a valve mechanism 314 through a tube 316 connected to a first lumen (not shown) of biluminal catheter 302 at a junction 318. A gas port 320 is connected to a second lumen (not shown) of biluminal catheter 302 at junction 318 by a tube 322. It is desirable to minimize the length of the tube 322 to minimize flow resistance.

Once inserted into the body cavity in which laparoscopic surgery is to be performed, balloon 304 of biluminal catheter 302 may be inflated, and slotted inserter 310 may be withdrawn. Once balloon 304 is inflated, biluminal catheter 302 may be drawn back until inflated balloon 304 seals against the inner surface 324 of the body cavity wall 308. This forms a relatively vapor tight seal. The puncture through body cavity wall 308 through which biluminal catheter 302 was inserted closes around an outer surface of the biluminal catheter 302. Once this seal is formed, insufflation gas, typically CO₂ may be injected into the body cavity from gas port 320 via a second lumen of the biluminal catheter 302.

Referring to FIGS. 5b and 5c, the slotted insertion 310 is of no further use during the laparoscopic surgery once biluminal catheter 302 is inserted through body cavity wall. Consequently, it would be desirable to get inserter 310 out of the way. To accomplish this, inserter 310 may be formed with a slit 326 along a major axis thereof that allows biluminal catheter 302 to separate inserter 310 along slit 326 by exerting pressure on thinned region 332 from within inserter 310 allowing inserter 310 to readily be removed from biluminal catheter 302.

In FIG. 5c, an alternate embodiment of a mechanism for separating inserter 310 along a major axis thereof is shown. A tab 328 may be attached to a filament 330 embedded in thinned region 332 of inserter 310. Filament 330 is shown vertically offset from thinned region 332 for clarification. In practice, filament 330 is preferably coincident with a center of thinned region 332. It will be recognized that a thinned region 332 may be sufficient to remove inserter 310 without need of pull tab 328 and filament 330.

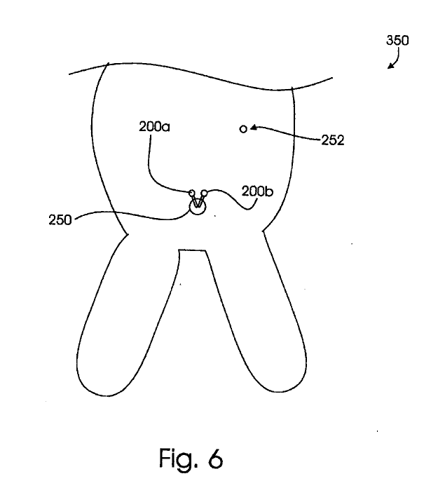

FIG. 6 is a schematic representation of a portion of a human abdomen, generally at reference number 350. A pair of trocars 200a, 200b is shown inserted into an abdominal cavity, preferably through a single incision in the umbilicus. A gas injection point 252 is shown displaced from umbilicus 250.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. An expandable trocar comprising:
a) a first elongated semi-circular section having a first flared proximal end, a first tapered distal end, a first major axis and a first pair of edges parallel to said major axis in at least a portion of a region between said first flared proximal ends and said first tapered distal end;
b) a second elongated semi-circular section having a second flared proximal end, a second tapered distal end, a second major axis and a second pair of edges parallel to said second major axis in at least a portion of a region between said second flared proximal ends and said second tapered distal end;
c) a plurality of outwardly projecting ribs disposed on an outside surface of said first semi-circular section, each of said plurality of ribs extending at least a portion of the circumferential distance between a first of said first pair of edges and a second of said first pair of edges; and
d) a plurality of outwardly projecting ribs disposed on an outside surface of said second semi-circular section, each of said plurality of ribs extending at least a portion of the circumferential distance between a first of said second pair of edges and a second of said second pair of edges;
wherein said first semi-circular section is circumferentially engageable with said second semi-circular section to form a hollow tubular structure having a flared proximal end and a tapered distal end.

2. The trocar as recited in claim 1, wherein said circumferential engagement comprises inserting both of a first set of edges of said first semi-circular section into an interior region of said second semi-circular section between said second set of edges thereof.

3. The trocar as recited in claim 1, wherein said circumferential engagement comprises inserting both of a second set of edges of said second semi-circular section into an interior region of said first semi-circular section between said first set of edges thereof.

4. The trocar as recited in claim 1 wherein each of said first semi-circular section and said second semi-circular section is formed from a thin polymeric material.

5. The trocar as recited in claim 1, wherein said plurality of outwardly projecting ribs are disposed circumferentially and parallel to one another.

6. The trocar as recited in claim 5, wherein said plurality of outwardly projecting ribs are disposed in one of the orientations chosen from the group: perpendicular to a major axis of one of said first semi-circular section and said second semicircular section, and at an acute angle relative to a major axis of one of said first semi-circular section and said second semi-circular section.

7. The trocar as recited in claim 1, wherein said a plurality of outwardly projecting ribs disposed on an outside surfaces of both said first semi-circular section and said second semicircular section are disposed in a spiral.

8. The trocar as recited in claim 1, wherein said hollow tubular structure has a variable outside diameter and a corresponding variable inside diameter.

9. The trocar as recited in claim 8, wherein said variable outside diameter and said corresponding variable inside diameter are determined by the circumferentially engaged relationship between said first semi-circular section and said second semi-circular section.

10. The trocar as recited in claim 8, wherein said variable outside diameter and said corresponding variable inside diameter may be enlarged by exerting pressure on at least one of a inside surface of said first semi-circular section and said second semi-circular section by insertion of an object having a larger diameter than said variable inside diameter.

11. The trocar as recited in claim 1, further comprising:
e) a valve/fulcrum disposed within said tubular structure between said flared proximal end and said tapered distal end.

12. The trocar as recited in claim 11, wherein said valve/fulcrum is disposed approximately midway between said flared proximal end and said tapered distal end.

13. The trocar as recited in claim 11, wherein said valve/fulcrum comprises at least one of the materials and structures selected from the group: a single layer resilient material, a resilient member with a second layer, and a sponge material.

14. The trocar as recited in claim 11, wherein said valve/fulcrum comprises a sponge material that comprises an anti-fogging material.

15. The trocar as recited in claim 1, further comprising:
e) an obturator comprising a head, an elongated shaft having a proximal end attached to and extending outwardly away from a rear face of said head, and a pointed distal end, said rear face of said head having an indentation sized and configured to receive and retain an outer edge of said flared proximal end.

16. A method of performing single incision laparoscopic surgery, the method:
a) inserting at least two trocars through an incision into a body cavity of a patient;
b) providing insufflation gas to said body cavity through an angiocath located remotely from said at least two trocars;
c) inserting an optical instrument into said body cavity through a first one of said at least two trocars;
d) inserting a surgical instrument into said body cavity through a second one of said at least two trocars; and
e) manipulating said surgical instrument to perform a surgery.

17. A method of performing single incision laparoscopic surgery according to claim 16, further comprising f) removing an object through said second one of said at least two trocars, wherein pressure on an interior wall of said trocar causes said second one of said at least two trocars to expand diametrically to allow outward passage of said object through said second one of said at least two trocars.

18. An expandable trocar comprising:
a first semicircular section formed of a resilient material; and
a second semicircular section formed of the resilient material and cooperably engaged with the first semicircular section to define a hollow trocar stem having a cross-sectional inside area,
wherein the first semicircular section is movable relative to the second semicircular section to expand the cross-sectional inside area of the hollow trocar stem while maintaining the cooperative engagement between the first and second semicircular sections.

19. An expandable trocar according to claim 18, wherein the first and second semicircular sections define a flared proximal end and a tapered distal end.

20. An expandable trocar according to claim 19, further comprising a valve/fulcrum positioned within the hollow trocar stem, the valve/fulcrum being configured to prevent gas from escaping through the hollow trocar stem and to support the trocar at an incision point.
